(19) United States
(12) Patent Application Publication
(10) Pub. No.: US 2004/0153093 A1
(43) Pub. Date: Aug. 5, 2004

(54) Bi-manual phacoemulsification apparatus and method

(75) Inventor: Stephen Donovan, Capistrano Beach, CA (US)

Correspondence Address: Peter J. Gluck, Advanced Medical Optics, Inc., 1700 E. St. Andrew Place, Santa Ana, CA, 92705, US

(73) Assignee: ADVANCED MEDICAL OPTICS, INC., Santa Ana, CA

(21) Appl. No.: 10/355,844

(22) Filed: Jan. 31, 2003

(51) Int. Cl. A61B 17/20

(52) U.S. Cl. 606/108; 606/171; 604/22

(57) ABSTRACT

Apparatus for the removal of intraocular tissue from an eye includes an unsleeved vibrated needle insertable through a first eye incision into the intraocular tissue for emulsifying same. The needle is ultrasonically vibrated for emulsifying the intraocular tissue in a manner preventing substantial heat damage to the eye and a combined aspiration/irrigation needle or separate aspiration and irrigation needles are provided for both introducing irrigation fluid into the eye and aspirating emulsified intraocular tissue and irrigation fluid from the eye.

The present invention generally relates to apparatus and procedures with regard to ophthalmology and is more particularly directed to apparatus and methods for emulsifying and aspirating ocular tissue through the use of ultrasound. The present apparatus and method provides effective removal of intraocular tissue such as, for example, cataractous lens tissue, vitreous, blood clots, fibrin membrane and exudates.

With specific reference to lens removal, cataracts cause the lens of an eye to become clouded and a conventional practice to alleviate this condition is to surgically remove the cataractic lens and replace it with an artificial intraocular lens.

Original procedures, for example, cryophakia, required an eye incision of up to about 12 mm in length in order to enable the introduction of a needle or an instrument into the eye for manual extraction. Such devices, such as vitreotomes, included a working tip shaped as two co-axially arranged tubes with cutting edges. Tissue to be removed is aspirated into a passage between the tubes after being subjected to a shearing type cutting action.

On the other hand, phacoemulsification procedures enable the removal of a cataractic lens through a much smaller incision, for example, between about 2.5 to about 4 mm. In this procedure, a needle is inserted through the incision into a lens capsule and the needle is ultrasonically vibrated to mechanically emulsify the lens. Once fragmented, or emulsified, the lens material is aspirated through a lumen through the phacoemulsification needle.

During emulsification of the lens and aspiration of lens fragments, a flow of irrigation fluid is introduced into the lens capsule via an annulus established by a sleeve concentrically disposed over the needle. This flow of liquid into the eye is necessary in order to prevent collapse of the interior chamber of the eye during aspiration. Further, the irrigation fluid cools the needle in order to prevent any thermal damage through the incised cornea or sclera tissue.

Thus, a conventional phacoemulsification needle includes a lumen therethrough for aspiration and a sleeve for providing an annulus to introduce irrigation fluid into the eye. This construction unfortunately increases the incision size necessary for insertion of the needle.

Attempts to utilize smaller phacoemulsification needles results in very fine, i.e. narrow, passages for the irrigation fluid and aspirated tissue. This may result in needle blockage by large fragments of emulsified lens. It should be appreciated that such blockage produces increased vacuum and a disruption in fluid flow, which can affect intraocular fluid balance and overheating of the needle due to reduced fluid flow.

The present invention overcomes the disadvantages of the prior art through the utilization of an unsleeved, or bare, phacoemulsification needle. This enables a much smaller incision than heretofore possible.

### SUMMARY OF THE INVENTION

In accordance with one embodiment of the present invention, apparatus is provided for the removal of intraocular tissue. The apparatus includes an unsleeved vibrated needle insertable through a first eye incision into the intraocular tissue for emulsifying the intraocular tissue.

The apparatus further includes means for providing ultrasonic energy to the vibrated needle effective for emulsifying the intraocular tissue in a manner preventing substantial heat damage to the said eye and an aspiration/irrigation needle is provided for insertion through a second eye incision for providing irrigation fluid into the eye and aspirating emulsified intraocular tissue and the irrigation fluid from the eye.

The invention is suitable for removal of intraocular tissue such as cataractous lens tissue, vitreous, blood clots, fibrin membrane and exudates.

In order to minimize the size of the first eye incision, the unsleeved vibrated needle may have a solid cross-section.

In an alternative embodiment of the present invention, an unsleeved vibrated needle is provided for insertion through a first eye incision into intraocular tissue, which may be cataractous lens tissue, for emulsifying same.

The apparatus further includes means for providing ultrasonic energy to the vibrated needle effective for emulsifying the tissue in a manner preventing substantial heat damage to the eye.

More particularly, an irrigation needle is provided and insertable through a second eye incision for providing irrigation fluid into the eye and an aspiration needle is provided and insertable through a third eye incision for aspirating emulsified intraocular tissue and irrigation fluid from the eye. In this embodiment, because of three separate needles are utilized, each of the first, second and third eye incisions may be made smaller than heretofore required.

A method in accordance with the present invention includes the steps of making first and second incisions into an eye and thereafter inserting an unsleeved needle through the first incision into intraocular tissue and inserting an aspiration/irrigation needle through the second incision.

The unsleeved needle is vibrated in a manner effective for emulsifying the intraocular tissue while preventing substantial heat damage to the eye. Irrigation fluid is provided through the aspiration/irrigation needle and emulsified intraocular tissue and irrigation fluid are aspirated from the eye through the aspiration/irrigation needle.

The method may further comprise a step of selecting intraocular tissue from a group of tissue consisting of cataractous lens tissue, vitreous, blood clots, fibrin membrane and exudates. In this method, the step of making the first incision may comprise of making the incision of the maximum dimension of about 2.0 mm. In addition, the step of making the second incision may comprise making the second incision with a maximum dimension of about 1.5 mm.

In an alternative embodiment of the present invention, a method is provided for the removal of intraocular tissue, which includes making first, second and third incisions into an eye and thereafter inserting a unsleeved needle through the first incision intraocular tissue, inserting an irrigation needle through the second incision, and inserting an aspiration needle through the third incision.

The unsleeved needle is then vibrated in a manner effective for emulsifying the intraocular tissue while preventing substantial heat damage to the eye. Irrigation is provided into the eye through the irrigation needle and emulsified intraocular tissue and irrigation fluid is aspirated from the eye through the aspiration needle.

In this embodiment, the method includes making the first incision with a maximum dimension of about 2.0 mm and making the second and third incisions with maximum dimensions of about 1.5 mm, and 1.5 mm respectively.

### BRIEF DESCRIPTIONS OF THE DRAWINGS

The advantages and features of the present invention will be better understood by the following description when considered in conjunction with the accompanying drawings in which:

FIG. 1 is a diagram of one embodiment of the present invention generally showing an unsleeved needle, insertable through a first eye incision for emulsifying intraocular tissue, a means for providing ultrasonic energy to the vibrated needle and an aspiration/irrigation needle, insertable through a second eye incision, for providing both irrigation fluid into the eye and aspirating emulsified intraocular tissue and the irrigation fluid from the eye;

FIG. 2 is a side view of the unsleeved needle shown in FIG. 1, which may be solid in cross-section in order to minimize incision size;

FIG. 3 is a diagram of an alternative embodiment of the present invention generally showing an unsleeved vibrated needle, insertable through a first eye incision, for emulsifying intraocular tissue, means for providing ultrasonic energy to the vibrated needle, an irrigation needle, insertable through a second eye incision, for providing irrigation fluid into the eye and an aspiration needle, insertable through a third eye incision, for aspirating emulsified intraocular tissue and the irrigation fluid from the eye.

### DETAILED DESCRIPTION

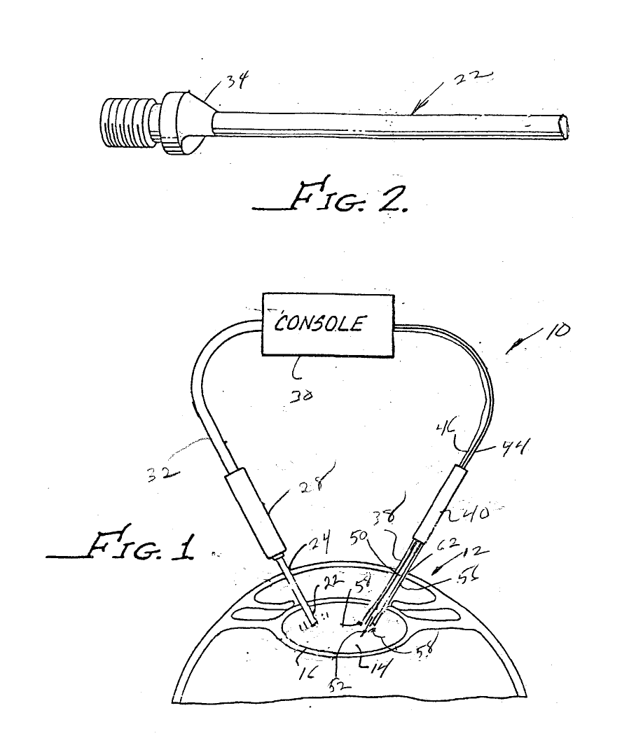

With reference to FIG. 1, there is shown apparatus 10 for the removal of intraocular tissue from an eye 12 specifically illustrating the use of the apparatus 10 for the removal of cataractous tissue 14 from an eye capsule 16.

The apparatus 10 includes an unsleeved vibrated needle 22, which is insertable through a first eye incision 24 for emulsifying the lens tissue 14.

Manipulation of the needle 22 is effected through a handpiece 28, which communicates with a control console 30 through a line 32.

The console 30 is a means for providing ultrasonic energy to the vibrated needle 22 in a manner preventing damage to the first eye incision 24 by heating thereof.

Suitable console means 30 is disclosed in co-pending U.S. patent application Ser. No. 10/278,775 filed on Oct. 21, 2002 entitled “NOVEL ENHANCED MICROBURST ULTRASONIC POWER DELIVERY SYSTEM AND METHOD.”This application is to be incorporated in its entirety by this specific reference thereto as describing the means for providing ultrasonic energy to the vibrated needle 22 effective for emulsifying the intraocular tissue 14 in a manner preventing substantial heat damage to the eye particularly tissue at the first incision 24. It is well known that the onset of thermal, or heat, damage to an eye, i.e. eye tissue, beings at between about 41°-42° C. and substantial heat damage can occur at about 45° C. In the present invention, the apparatus and method limits the elevation of eye tissue temperature to less than about 45° C. and preferably to less than about 41°-42° C.

Because the needle 22 is unsleeved, i.e. bare, the first incision 24 may be as small as 0.8 mm or less which is to be compared with conventional sleeved needles (not shown) which require an incision opening having dimensions of about 2½ to 3 mm.

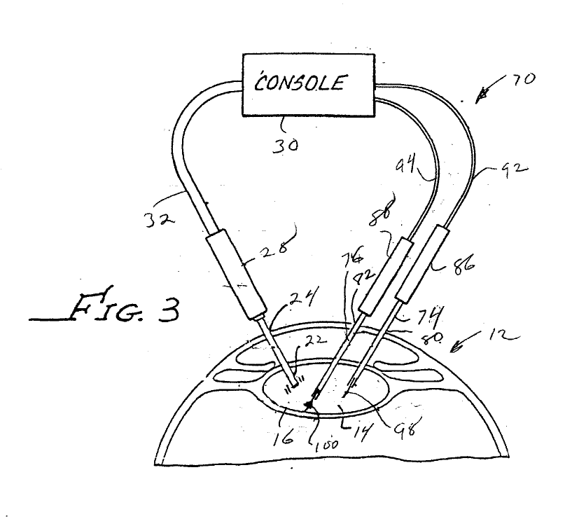

As most clearly shown in FIG. 2, the needle 22 may be solid and includes a conventional hub 34 for attachment of the needle 22 to the handpiece 28.

With reference again to FIG. 1, the apparatus 10 includes an aspiration/irrigation needle 38 manipulated by a handpiece 40, which is in turn interconnected to the console 30 by irrigation and aspiration lines 44, 46 respectively. An inner lumen of the needle 38 provides irrigation fluid into the eye as indicated by the arrow 52 and an annulus 56 enables aspiration of emulsified intraocular tissue 14 and irrigation fluid from the eye as indicated by the arrows 58.

Irrigation fluid and aspiration vacuum is provided to the handpiece 40 and needle 38 in a conventional manner by the console 30. Because a vibrated needle (not shown) is not present in the aspiration, an irrigation needle 38 a second eye incision 62 through which the needle 38 is inserted may be made as small as 0.8 mm or less. Thus, the overall trauma to the eye 12 is minimized through use of the apparatus 10 in accordance with the present invention.

With reference to FIG. 3, there is shown an alternative embodiment 70 of the present invention utilizing a separate irrigation needle 74 and separate aspiration needle 76, insertable through second and third eye incisions 80, 82 respectively. Common reference characters shown in FIG. 3 refer to identical or substantially similar components hereinabove discussed in connection with the embodiment 10 shown in FIG. 1.

The irrigation needle 74 is manipulated by an irrigation handpiece 86 and the aspiration needle 76 is manipulated by an aspiration handpiece 88 with the handpieces 88, 86 being interconnected to the console 30 in a conventional manner through an irrigation line 92 and an aspiration line 94. Coordination of fluid flows is made in a conventional manner through the console 30. Irrigation fluid is represented by the arrow 98 is provided to maintain intraocular pressure while at the same time facilitating aspiration of emulsified lens tissue through the aspiration needle as represented by the arrow 100.

It should be appreciated that the FIGS. 1 and 3 are diagrams of the apparatus 10, 70 in accordance with the present invention and the placement of the needles 22, 38, 74, 76 is not to be construed as illustrating the actual placement and manipulation of the needles during a phacoemulsification procedure, but only as shown in an illustrative manner for explaining and describing the components of the present invention.

With regard to the method of operation, the present invention provides for the removal of intraocular tissue, which include the steps of making the first and second incisions 24, 64 into the eye and inserting the unsleeved needle 22 through the first incision 24 into the intraocular tissue 14.

The aspiration/irrigation needle 38 is inserted through the second incision 62 and the unsleeved needle 22 is vibrated in a manner effective for emulsifying the intraocular tissue 14 while preventing damage to the first incision 24 by heating thereof.

Irrigation fluid is indicated by the arrow 52 is provided into the eye 12 through the aspiration/irrigation needle 38 and emulsified intraocular tissue 14 and irrigation fluid are aspirated from the eye 12 through the aspiration/irrigation needle 38, as indicated by the arrows 58.

The method of utilization of the embodiment 70 shown in FIG. 3 includes the steps of making a first, second and third incisions 24, 80, 82 into the eye 12 and thereafter inserting the unsleeved needle 22, irrigation needle 74 and aspiration needle 76 through the respective incisions 24, 80 and 82.

The unsleeved needle 22 is vibrated as hereinabove described and irrigation fluid is provided into the eye through the irrigation needle 74 and emulsified intraocular tissue and irrigation fluid are aspirated from the eye 12 through the aspiration needle 76. In this method, the first, second and third incisions 24, 80, 82 may have dimensions of between about 0.8 mm to about 2.0 mm or less, and preferably less than about 1 mm.

Although there has been hereinabove described a specific bi-manual phacoemulsification apparatus and method in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclose herein. Accordingly, any and all modifications, variations or equivalent arrangements which may occur to those skilled in the art, should be considered to be within the scope of the present invention as defined in the appended claims.

What is claimed is:

1. Apparatus for the removal of intraocular tissue from an eye, said apparatus comprising:
an unsleeved vibrated needle insertable through a first eye incision into said intraocular tissue for emulsifying said intraocular tissue;
means for providing ultrasonic energy to said unsleeved vibrated needle effective for emulsifying said intraocular tissue in a manner preventing substantial heat damage to said eye; and
an aspiration/irrigation needle insertable through a second eye incision for providing both irrigation fluid into said eye and aspirating emulsified intraocular tissue and the irrigation fluid from the eye.

2. The apparatus according to claim 1 wherein said intraocular tissue is selected from a group consisting of cataractous lens tissue, vitreous, blood clots, fibrin membrane and exudates.

3. The apparatus according to claim 1 wherein said unsleeved vibrated needle has a solid cross-section.

4. Apparatus for the removal of intraocular tissue from an eye, said apparatus comprising:
an unsleeved vibrated needle insertable through a first eye incision into said intraocular tissue for emulsifying said intraocular tissue;
means for providing ultrasonic energy to the vibrated needle effective for emulsifying said intraocular tissue in a manner preventing substantial heat damage to said eye;
an irrigation needle insertable through a second eye incision for providing irrigation fluid into said eye; and
an aspiration needle insertable through a third eye incision for aspirating emulsified intraocular tissue and the irrigation fluid from the eye.

5. The apparatus according to claim 4 wherein said intraocular tissue is selected from a group consisting of cataractous lens tissue, vitreous, blood clots, fibrin membrane and exudates.

6. The apparatus according to claim 4 wherein said unsleeved vibrated needle has a solid cross-section.

7. Apparatus for the removal of lens tissue from a lens capsule, said apparatus comprising:
an unsleeved vibrated needle insertable through a first incision into said lens capsule for emulsifying lens tissue;
means for providing ultrasonic energy to the vibrated needle effective for emulsifying said lens tissue in a manner preventing substantial heat damage to first; and
an aspiration/irrigation needle insertable through a second incision into said lens capsule for both providing irrigation fluid into said lens capsule and aspirating emulsified lens tissue and irrigation fluid from said lens capsule.

8. The apparatus according to claim 7 wherein said unsleeved vibrated needle has a solid cross-section.

9. Apparatus for the removal of lens tissue from a lens capsule, said apparatus comprising:
an unsleeved vibrated needle insertable through a first incision into said lens capsule for emulsifying lens tissue;
means for providing ultrasonic energy to the vibrated needle effective for emulsifying said lens tissue in a manner preventing substantial heat damage to said eye;
an irrigation needle insertable through a second incision into said lens capsule for providing irrigation fluid into said lens capsule; and
an aspiration needle insertable through a third incision into said lens capsule for aspirating emulsified lens tissue and the irrigation fluid from said lens capsule.

10. The apparatus according to claim 9 wherein said unsleeved vibration needle has a solid cross-section.

11. A method for the removal of intraocular tissue from an eye, the method comprising the steps of:
making a first incision into said eye;
making a second incision into said eye;
inserting an unsleeved needle through said first incision into said intraocular tissue;
inserting an aspiration/irrigation needle through said second incision;
vibrating said unsleeved needle in a manner effective for emulsifying said intraocular tissue while preventing substantial heat damage to said eye;
providing irrigation fluid into said eye through said aspiration/irrigation needle; and
aspirating emulsified intraocular tissue and the irrigation fluid from said eye through said aspiration/irrigation needle.

12. The method according to claim 11 wherein said method further comprises selection of intraocular tissue from a group consisting of cataractous lens tissue, vitreous, blood clots, fibrin membrane and exudates.

13. The method according to claim 11 wherein the step of making the first incision comprises the first incision with a maximum dimension of about 2.0 mm.

14. The method according to claim 12 wherein the step of making the second incision comprises making the second incision with a maximum dimension of about 1.5 mm.

15. A method for the removal of intraocular tissue from an eye, the method comprising the steps of:
making a first incision into said eye;
making a second incision into said eye;
making a third incision into said eye;
inserting an unsleeved needle through said first incision into said intraocular tissue;
inserting an irrigation needle through said second incision;
inserting an aspirating needle through said third incision;
vibrating said unsleeved needle in a manner effective for emulsifying said intraocular tissue while preventing substantial heat damage to said eye;
providing irrigation fluid into said eye through said irrigation needle; and
aspirating emulsified intraocular tissue and the irrigation fluid from said eye through said aspiration needle.

16. The method according to claim 15 wherein said method further comprises selection of intraocular tissue from a group consisting of cataractous lens tissue, vitreous, blood clots, fibrin membrane and exudates.

17. The method according to claim 15 wherein the step of making the first incision comprises the first incision with a maximum dimension of about 2.0 mm.

18. The method according to claim 17 wherein the step of making the second incision comprises making the second incision with a maximum dimension of about 1.5 mm.

19. The method according to claim 18 wherein the step of making the third incision comprising making the third incision with a maximum dimension of about 1.5 mm.
